SEQUENCE LISTING (12 sequences)

SEQ ID NO: 1 - DNA, 19 bp, artificial sequence
  Oligonucleotide primer
tactgggcctcagccaact

SEQ ID NO: 2 - DNA, 21 bp, artificial sequence
  Oligonucleotide primer
gaaggattttgaagcggagaa

SEQ ID NO: 3 - DNA, 21 bp, artificial sequence
  Oligonucleotide probe
cctcagtcccagccagccatg

SEQ ID NO: 4 - DNA, 21 bp, artificial sequence
  Oligonucleotide primer
acagatgaagtgctccttcca

SEQ ID NO: 5 - DNA, 21 bp, artificial sequence
  Oligonucleotide primer
gtcggagattcgtagctggat

SEQ ID NO: 6 - DNA, 20 bp, artificial sequence
  Oligonucleotide probe
ctctgccctctggatggcgg

SEQ ID NO: 7 - DNA, 22 bp, artificial sequence
  Oligonucleotide primer
tgtctgtctgcagattggctac

SEQ ID NO: 8 - DNA, 20 bp, artificial sequence
  Oligonucleotide primer
agatggatcagccaagaagg

SEQ ID NO: 9 - DNA, 25 bp, artificial sequence
  Oligonucleotide probe
cggattacaccttcccacttgctga

SEQ ID NO: 10 - DNA, 16 bp, artificial sequence
  Oligonucleotide primer
cctttgggctggtcat

SEQ ID NO: 11 - DNA, 17 bp, artificial sequence
  Oligonucleotide primer
gcacttcagggttgtag

SEQ ID NO: 12 - DNA, 22 bp, artificial sequence
  Oligonucleotide probe
ccagcaggtgttcgacaatggc

(19) United States
(12) Patent Application Publication
(10) Pub. No.: US 2012/0190031 A1
(43) Pub. Date: Jul. 26, 2012

(54) MARKER FOR DIAGNOSIS OF ACTIVE MULTIPLE SCLEROSIS

(75) Inventors: Patrick Stordeur, Forest (BE); Françoise Villée, Ville-pommeroeul (BE); Mathieu Vokaer, Forest (BE); Annick Ocmant, Le Roeulx (BE); Michel Goldman, Uccle (BE)

(73) Assignee: UNIVERSITÉ LIBRE DE BRUXELLES, Bruxelles (BE)

(21) Appl. No.: 13/203,531

(22) PCT Filed: Mar. 25, 2010
(86) PCT No.: PCT/EP2010/053944

§ 371 Date: Aug. 26, 2011

(30) Foreign Application Priority Data
Mar. 27, 2009 (EP) 09156388.2

(51) Int. Cl. C12Q 1/68

(52) U.S. Cl. 435/6.12

(57) ABSTRACT

The invention provides an improved method for monitoring the course of the MS disease and to predict, diagnose or prognosticate whether a subject is in an active or non-active period of MS. The method is based on determining the ratio of the expression levels of two cytokines, measured before and after stimulation of the subject by an immunomodulator.

### FIELD OF THE INVENTION

The present invention is directed to the medical field. In particular, the present invention is directed to a method for diagnosing, predicting or prognosticating whether an individual is going through an active phase of multiple sclerosis (MS), i.e. is at risk of having an attack or of a relapse. The method is based on the determination of cytokine mRNA levels in blood samples from patients before and after injection with an immunomodulator of the type I interferon type or a related stimulating agent.

### BACKGROUND OF THE INVENTION

Multiple sclerosis (MS) is a chronic, inflammatory disease that affects the central nervous system (CNS). MS can cause a variety of symptoms, including changes in sensation, visual problems, muscle weakness, depression, difficulties with coordination and speech, severe fatigue, and pain. Although many patients lead full and rewarding lives, MS can cause impaired mobility and disability in more severe cases.

Multiple sclerosis affects neurons, the cells of the brain and spinal cord that carry information, create thought and perception, and allow the brain to control the body. Surrounding and protecting some of these neurons is a fatty layer known as the myelin sheath, which helps neurons carry electrical signals in much the same way as the isolation layer of electric wiring. MS causes gradual destruction of myelin (demyelination) and transection of neuron axons in patches throughout the brain and spinal cord, causing signals to get patched through to the wrong place or stopping the signaling. The name multiple sclerosis refers to the multiple scars (or scleroses) on the myelin sheaths. This scarring causes symptoms which vary widely depending upon which signals are interrupted. It is thought that MS results from attacks by an individual's immune system on the nervous system and is therefore categorized as an autoimmune disease. MS currently does not have a cure, though several treatments are available which may slow down the appearance of new symptoms. Interferon-beta (IFN-beta), a type-I Interferon, is a pleiotropic cytokine with immunomodulatory properties and has become a global standard in the treatment of MS. Despite the well documented efficacy in responders to this medication, a substantial number of patients fail to respond to IFN-beta. Why IFN-beta therapy is or is not effective with respect to MS, and how IFN-beta alters the clinical course of MS, remain unclear. Putative mechanisms of action include the inhibition of T cell proliferation, regulation of a large number of cytokines, and blocking of blood-brain barrier opening via interference with cell adhesion, migration and matrix metalloproteinase activity.

The majority of MS patients present a relapsing-remitting clinical course. Symptoms of MS usually appear in episodic acute periods of worsening, also called relapses, exacerbations, bouts or attacks, in gradually-progressive deterioration of neurologic function, or in a combination of both. The person with MS can suffer almost any neurological symptom or sign, including changes in sensation (hypoesthesias and paraesthesias), muscle weakness, muscle spasms, or difficulty in moving, difficulties with coordination and balance (ataxia), problems in speech (dysarthria) or swallowing (dysphagia), visual problems (nystagmus, optic neuritis, or diplopia), fatigue, acute or chronic pain, and bladder and bowel difficulties. Cognitive impairment of varying degrees and emotional symptoms of depression or unstable mood are also common (Lublin and, Reingold 1996, Neurology 46 (4): 907-11).

The main clinical measure of disability progression and symptom severity is called the Expanded Disability Status Scale or EDSS (Kurtzke 1983, Neurology 33 (11): 1444-52). Although some attacks are preceded by common triggers such as the common cold, influenza infection, gastroenteritis, stress, etc., MS relapses are often unpredictable, occurring without warning and without obvious inciting factors. The prognosis of the disease (the expected future course of the disease) for a person with multiple sclerosis depends on the subtype of the disease, the individual's sex, age, and initial symptoms and the degree of disability the person experiences and is thus very difficult to establish (Weinshenker 1994, Ann. Neurol. 36 Suppl: S6-11).

Currently there is no clear and easy to use predictive or diagnostic method to establish whether a subject is going through an active period of MS or not.

It is therefore an aim of the present invention to provide an improved method for monitoring the course of the MS disease and to predict, diagnose or prognosticate whether a subject is in an active or non-active period of MS, which overcomes at least some of the above-mentioned drawbacks of known methods. This is important to know in view of precautionary measures to be taken and determining suitable treatments, in order to avoid or at least monitor and control relapses or attacks.

### SUMMARY OF THE INVENTION

In search for a tool or method to distinguish patients having active MS from patients having non-active MS, the inventors investigated whether it was possible to discriminate between both active states, using the expression level of one or more cytokines.

Although it is well known that several cytokines may be implicated in both the development and disease course of MS, the inventors unexpectedly found a correlation between the ratio of the stimulation index of two specific cytokines, namely IL-23p19 and IL-1-beta, and the active state of MS in the tested subjects. With stimulation index, the inventors mean the ratio of the expression level of the cytokine after and before treatment of the patient with an immunomodulator such as e.g. type I IFN.

In particular, the inventors found unexpectedly that there is a clear correlation between the equation R and the active state of MS, wherein equation R is defined being equal to Stimulation index IL-23p19 divided by Stimulation index IL-1-β.

More particularly, the correlation is calculated using the following equation:

$$R 1 = \frac{\frac{Blood mRNA levels of IL \text{-} 23 p 19 after stimulation}{Blood mRNA levels of IL \text{-} 23 p 19 before stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index IL \text{-} 23 p 19 ) \\ ( Stimulation index IL \text{-} 1 \text{-} β )\end{matrix}$$

The invention thus provides a method for predicting, diagnosing and/or prognosticating Multiple Sclerosis (MS) in a subject, comprising the steps of:

(i) measuring the level of IL-23p19 in the sample from the subject before and after stimulation with an immunomodulator, yielding a stimulation index value for IL-23p19;

(ii) measuring the level of IL-1-beta in the sample from the subject before and after stimulation with an immunomodulator, yielding a stimulation index value for IL-1-beta;

(iii) correlating the relation between the two stimulation indexes obtained in steps (i) and (ii) with respect to each other with the activity status of MS in the subject.

In a preferred embodiment, the correlation step is done using the equation:

R=Stimulation index IL-23p19/Stimulation index IL-1-beta, or any rearrangement thereof.

In a further preferred embodiment, the correlation step is done using the equation:

$$R 1 = \frac{\frac{level of IL \text{-} 23 p 19 after stimulation}{level of IL \text{-} 23 p 19 before stimulation}}{\frac{level of IL \text{-} 1 \text{-} beta after stimulation}{level of IL \text{-} 1 \text{-} beta before stimulation}} \begin{matrix}( Stimulation index IL \text{-} 23 p 19 ) \\ ( Stimulation index IL \text{-} 1 \text{-} beta )\end{matrix}$$

or any rearrangement thereof.

When the equation R1 is used, an increased value of R1 when compared to a reference value is indicative for active MS in the subject.

wherein the first and second blood samples are preferably stabilized with a stabilizing agent, as fast as possible after blood collection.

In a preferred embodiment of the invention, the reference value of R1 is 2.0, and subjects having a value of R1 of 2 or more are classified as being in a period of active MS.

In a preferred embodiment of the invention, the reference value of R1 is 1.0, and subjects having a value of R1 of 1 or more are classified as being at risk of being in a period of active MS or evolving to a period of active MS.

In a preferred embodiment of the invention, the reference value of R1 is 1.0, and subjects having a value of R1 of less than 1.0 are classified as highly likely being in a period of inactive MS.

In one embodiment, the stimulation with an immunomodulator is, or has been done in vivo, i.e. by administering said immunomodulator to the subject under investigation e.g. by intraperitoneal, subcutaneous or intravenous injection or by oral administration. In an alternative embodiment, the stimulation with an immunomodulator is done in vitro, by adding said immunomodulator to the sample, after it was obtained from, or is taken from, the subject.

In a preferred embodiment of the method of the invention, the reference value of R1 is calculated based on the cytokine levels in a sample from a subject having non-active MS or no MS.

In a preferred embodiment of the method of the invention, the mRNA or protein level of the cytokines is determined.

In a preferred embodiment of the method of the invention, the sample is selected from the group consisting of: blood, whole blood, plasma or serum. Preferably, said sample is a whole blood sample.

In a preferred embodiment of the method of the invention, the subject is a human suffering from a disease which can be treated with a type I interferon, multiple sclerosis (MS), (chronic) hepatitis C (HCV) and/or B (HBV).

In a more preferred embodiment of the method of the invention, the immunomodulator is selected from the group consisting of: purified or recombinant type-1 interferon (type I IFN), such as IFN-alpha and IFN-beta, IFN-alpha-2a, IFN-alpha-2b, IFN-beta-1a, IFN-beta-1b; agents having similar effects or use in MS such as: Glatiramer Acetate, synthetic polypeptides with a structure resembling myelin, Natalizumab, anti-CD52, anti-CD25; agents acting on the sphingosine receptors such as FTY720; agents having an sequestering effect on lymphocytes; agents depleting T-lymphocytes; or Th2-cell response inducing agents such as Fumarate, and wherein the administration of the immunomodulator is performed by intraperitoneal, subcutaneous or intravenous injection or is administered orally.

The invention also provides a kit for diagnosing active MS in the subject comprising or consisting of:

(i) means for measuring the level of cytokine IL-23p19 in a sample of the subject;

(ii) means for measuring the level of cytokine IL-1-beta in a sample of the subject;

(iii) optionally an immunomodulator;

(iv) means and/or instructions for calculating the equation according to the method of the invention.

In a preferred embodiment of the kit of the invention, the means for determining either cytokine level is a means for determining the mRNA or protein level, such as end-point-PCR, real-time-PCR, quantitative-PCR, digital-PCR, or northern blot, capable of determining the mRNA level of the cytokines, or ELISA, ELISPOT, Luminex's xMAP technology, flow cytometry, nephelometry, turbidimetry, immunoprecipitation, capable of determining the concentration of cytokines.

In a more preferred embodiment of the kit of the invention, the kit comprises: a) one or more vessel(s) suitable for accepting a sample such as e.g. a blood sample, b) a primer pair specific to the mRNA of the IL-23p19 gene which is suitable for the transcription of mRNA of said control gene into cDNA and the amplification of the latter, and a probe designed to anneal to an internal region of the produced cDNA, c) a primer pair specific to the mRNA of the IL-1-beta gene which is suitable for the transcription of mRNA of said control gene into cDNA and the amplification of the latter, and a probe designed to anneal to an internal region of the produced cDNA, wherein said vessel comprises: (i) a vessel capable of accepting a blood sample, and optionally (ii) a container in which a stabilizing agent is present, (iii) a connection between the inside of said vessel (i) and the inside of said container (ii), and (iv) a physical barrier that temporarily blocks said connection (iii).

In a further preferred embodiment, the kit of the invention can further comprise a container (v) in which a sufficient amount of immunomodulator as defined herein is present, a connection of said container (v) to the sample vessel (i). This container (v) can again be separated from the sample vessel (i) by a physical barrier (vii) that temporarily blocks the connection (vi). Alternatively, said immunomodulator can be present in the sample vessel prior to the addition of the sample. In both embodiments, the immunomodulator can e.g. be present in powder, liquid or lyophilised form.

In another embodiment of the kit of the invention, means for determining either cytokine level is a specific binding assay, immunodetection assay, Mass-spectrometry assay, capable of determining the protein level of the cytokines.

In a more preferred embodiment of the kit of the invention, said immunomodulator is purified or recombinant type-1 interferon (type I IFN), such as IFN-alpha and IFN-beta, IFN-alpha-2a, IFN-alpha-2b, IFN-beta-1a, IFN-beta-1b; agents having similar effects or use in MS such as: Glatiramer Acetate, synthetic polypeptides with a structure resembling myelin, Natalizumab, anti-CD52, anti-CD25; agents acting on the sphingosine receptors such as FTY720; agents having an sequestering effect on lymphocytes; agents depleting T-lymphocytes; or Th2-cell response inducing agents such as Fumarate.

The invention further provides for the use of a kit according to any one of the herein defined embodiments, for diagnosing, predicting or prognosticating the active state of MS in a subject, preferably by performing the method according to the invention as defined herein.

The methods of the invention can alternatively be performed using a kit according to the invention.

In addition, the invention provides for the use of the methods and kits of the invention for monitoring the treatment of an MS patient, comprising performing the steps as defined herein at different time points during the treatment, wherein reduced ratios point to a reduction in MS activity in the subject under treatment, indicating the treatment is indeed beneficial for the subject.

Finally, the invention provides for the use of the methods and kits of the invention for determining the treatment needed for an MS patient comprising performing the method steps or using the kits as defined herein at different time points during the treatment, wherein increased ratios point to active MS in the subject under observation, indicating the need for active MS specific treatment.

Those skilled in the art will immediate recognize the many other effects and advantages of the present method and the numerous possibilities for end uses of the present invention from the detailed description and examples provided below.

### BRIEF DESCRIPTION OF THE DRAWINGS

FIG. 1: Schematic overview of the procedure

A first blood sample was taken from the patient, before treatment and stabilised. Patients were subsequently injected with type I IFN. 4 hours after the injection, a second blood sample was taken and stabilised. The mRNA level of two cytokines was determined by means of Q-PCR, in both samples. The Ratio R1 was calculated by dividing the IL-23p19 stimulation index value by the IL-1-beta stimulation index. The stimulation indexes are calculated by dividing the mRNA level after treatment by the mRNA level before treatment for each of the cytokines.

FIG. 2: Classifying patients based on the R value

IFN-b-treated multiple sclerosis (MS) patients were divided in two groups according to their disease status, i.e. clinically active or not, based on several parameters including EDSS score and MRI. For each patient, the ratio R1 was determined as explained above. This ratio R1 is higher in patients who are in active state compared with those which present an inactive disease, with a p value of 0.0001, which is highly significant (Mann Witney test).

FIG. 3: ROC curve

ROC analysis shows that this ratio has a poor sensitivity but a very good specificity. For example, for a cut-off value of 2.0, only 53% of the patients will be positive, but these positive patients will have 96% of likelihood to develop a relapse.

FIG. 4: ROC curve

The ROC curve of FIG. 3 presented in an alternative manner

FIG. 4: Ratio (R1) measured during treatment of patients. The disease activity is indicated as NA (non-active) or A (active). The number of days between taking the blood samples for analysis is also indicated. It is clear that the R1-value is increased in patients that go from a non-active MS to an active MS state, whereas in patients going from an active to a non-active MS state, the R1 value decreases.

### DETAILED DESCRIPTION OF THE PREFERRED EMBODIMENTS

Several subtypes of MS are known (Lublin and, Reingold 1996, Neurology 46 (4): 907-11). They are important for prognosis and for making therapeutic decisions. The United States National Multiple Sclerosis Society standardizes four subtype definitions: relapsing remitting, secondary progressive, primary progressive and progressive relapsing.

For 85-90% of individuals with MS the initial course is of the relapsing-remitting subtype, characterized by unpredictable relapses followed by periods of months to years of relative quiet (remission) with no new signs of disease activity. Deficits suffered during attacks may either resolve completely or incompletely. When deficits always resolve completely between the attacks, this is sometimes referred to as benign MS.

Secondary progressive MS describes those with initial relapsing-remitting MS, who then begin to have progressive neurologic decline between acute attacks without any definite periods of remission. Occasional relapses and minor remissions may appear. The median time between disease onset and conversion from relapsing-remitting to secondary progressive MS is 19 years.

The primary progressive subtype describes the approximately 10-15% of individuals who never have remission after their initial MS symptoms. It is characterized by progression of disability from onset, with no, or only occasional and minor, remissions and improvements. The age of onset for the primary progressive subtype is later than other subtypes.

Progressive relapsing MS describes those individuals who, from onset, have a steady neurologic decline but also suffer clear superimposed attacks. This is the least common of all subtypes.

Non-standard MS cases can include Devic's disease, Balo concentric sclerosis, Schilder's diffuse sclerosis and Marburg multiple sclerosis, although some are sometimes seen as being different diseases.

Although there is no known cure for multiple sclerosis, several therapies have proven helpful. The primary aims of therapy are returning function after an attack, preventing new attacks, and preventing disability. For managing acute attacks, the routine treatment is administration of high doses of intravenous corticosteroids, such as methylprednisolone. The aim of this kind of treatment is to end the attack sooner and leave fewer lasting deficits in the patient. Such treatments are generally effective in the short term but do not appear to help for long-term recovery. Some Disease-modifying treatments are being developed but are expensive and mostly require frequent (up-to-daily) injections. Examples are interferon beta-1a (Avonex® and Rebif®) and interferon beta-1b (Betaseron® and Betaferon®). A further example is glatiramer acetate (Copaxone). Also mitoxantrone, an immunosuppressant used in cancer chemotherapy is used for secondary progressive MS. Natalizumab (Tysabri®) is also used. The interferons and glatiramer acetate are delivered by frequent injections, varying from once-per-day for glatiramer acetate to once-per-week (but intra-muscular) for Avonex®. Natalizumab and mitoxantrone are given by intravenous (IV) infusion at monthly intervals. Treatment of progressive MS is more difficult than relapsing-remitting MS. Mitoxantrone has shown positive effects in patients with secondary progressive and progressive relapsing courses. It is moderately effective in reducing the progression of the disease and the frequency of relapses in patients in short-term follow-up. No treatment has been proven to modify the course of primary progressive MS. Disease-modifying treatments may reduce the progression rate of the disease, but do not provide a cure and as multiple sclerosis progresses, the symptoms tend to increase.

Known diagnostic methods are for example the Schumacher and Poser criteria (Poser 2004 Clin Neurol Neurosurg 106 (3): 147-58) and the McDonald criteria (McDonald et al., 2001, Ann. Neurol. 50 (1): 121-7). The most commonly used diagnostic tools are neuroimaging, analysis of cerebrospinal fluid and evoked potentials. Magnetic resonance imaging of the brain and spine shows areas of demyelination (lesions or plaques). Gadolinium can be administered intravenously as a contrast to highlight active plaques and, by elimination, demonstrate the existence of historical lesions not associated with symptoms at the moment of the evaluation (Rashid and Miller 2008 Semin Neurol 28 (1): 46-55). Testing of cerebrospinal fluid obtained from a lumbar puncture can provide evidence of chronic inflammation of the central nervous system. The cerebrospinal fluid is tested for oligoclonal bands, which are an inflammation marker found in 75-85% of people with MS (Link and Huang 2006, J. Neuroimmunol. 180 (1-2): 17-28). The nervous system of a person with MS often responds less actively to stimulation of the optic nerve and sensory nerves due to demyelination of such pathways. These brain responses can be examined using visual and sensory evoked potentials (Gronseth and Ashman 2000, Neurology 54 (9): 1720-5). The Kurtzke Expanded Disability Status Scale (EDSS) is a method of quantifying disability in multiple sclerosis (Kurtzke 1983, Neurology 33 (11):

1444-52). The EDSS quantifies disability in eight Functional Systems (FS) and allows neurologists to assign a Functional System Score (FSS) in each of these. EDSS steps 1.0 to 4.5 refer to people with MS who are fully ambulatory. EDSS steps 5.0 to 9.5 are defined by the impairment to ambulation.

The clinical meaning of each possible result is the following:
0.0: Normal Neurological Exam
1.0: No disability, minimal signs on 1 FS
1.5: No disability minimal signs on 2 of 7 FS
2.0: Minimal disability in 1 of 7 FS
2.5: Minimal disability in 2 FS
3.0: Moderate disability in 1 FS; or mild disability in 3-4 FS, though fully ambulatory
3.5: Fully ambulatory but with moderate disability in 1 FS and mild disability in 1 or 2 FS; or moderate disability in 2 FS; or mild disability in 5 FS
4.0: Fully ambulatory without aid, up and about 12 hrs a day despite relatively severe disability. Able to walk without aid 500 meters
4.5: Fully ambulatory without aid, up and about much of day, able to work a full day, may otherwise have some limitations of full activity or require minimal assistance. Relatively severe disability. Able to walk without aid 300 meters
5.0: Ambulatory without aid for about 200 meters. Disability impairs full daily activities
5.5: Ambulatory for 100 meters, disability precludes full daily activities
6.0: Intermittent or unilateral constant assistance (cane, crutch or brace) required to walk 100 meters with or without resting
6.5: Constant bilateral support (cane, crutch or braces) required to walk 20 meters without resting
7.0: Unable to walk beyond 5 meters even with aid, essentially restricted to wheelchair, wheels self, transfers alone; active in wheelchair about 12 hours a day
7.5: Unable to take more than a few steps, restricted to wheelchair, may need aid to transfer; wheels self, but may require motorized chair for full day's activities
8.0: Essentially restricted to bed, chair, or wheelchair, but may be out of bed much of day; retains self care functions, generally effective use of arms
8.5: Essentially restricted to bed much of day, some effective use of arms, retains some self care functions
9.0: Helpless bed patient, can communicate and eat
9.5: Unable to communicate effectively or eat/swallow
10.0: Death due to MS

The inventors have now unexpectedly found that determining the relation of the expression levels of two cytokines before and after stimulation with an immonomodulator can predict the active or non-active status of MS in a patient.

More in particular, the inventors have found that the following equation R is linked to the active status of the MS condition, wherein R is defined as:

R=Stimulation index of IL23p19/Stimulation index of IL-1-beta, wherein the stimulation index is the relative difference in expression level of each cytokine before and after stimulation with the immunomodulator.

More preferably, the equation used is as follows:

$$R 1 = \frac{\frac{Blood mRNA levels of IL \text{-} 23 p 19 after stimulation}{Blood mRNA levels of IL \text{-} 23 p 19 before stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index A ) \\ ( Stimulation index B )\end{matrix}$$

When determining the equation R1 as defined herein, typically values between 0.0 and 10.0 were obtained in the present set of patients.

The subjects having an R1-value of 2.0 or more, preferably 2.9 or more were all (i.e. 100%) found to be in the active MS state and thus at risk of having an attack or relapse. The cut-off value of 2.0 or more, preferably 2.9 or more is thus a 100% specific predictor for the active state of MS.

For subjects having an R1-value between 1.0 and 2.0, preferably between 1.0 and 2.9, the specificity is much lower, but still around 50%. An R1-value between 1.0 and 2.0, preferably between 1.0 and 2.9, should thus trigger a follow up of the patient since he may be at risk of having an attack.

For subjects with an R1-value below 1.0, the risk of having an attack or relapse is rather low, but not completely absent and these patients should also be monitored in the next few months, wherein a rise in the R1-value indicates the MS in the patient may be developing from non-active to active.

Some of the patients tested initially had non-active MS, but developed towards the active MS state during the course of the present analysis. It is very interesting to see that the R1-value of these patients doubled in the active versus non-active state of MS (cf. Table 1).

A clear rise in the R1-value is thus indicative of an increased risk of having an attack.

The exact mechanism clarifying the results of the resent invention is not known, but it is clear that the cytokines IL-23p19 and IL-1-beta are involved in the disease process. Analysing the expression levels of each of the cytokines individually does not result in any predictive power, but the relation between both cytokine stimulation indexes somehow does. This is of course rather unexpected, since although both cytokines are clearly involved in a Th-17 immune response, their expression level appears to be evolving at a different speed during the course of disease development, namely, the IL-1-beta expression is lowered much faster (almost twice as fast) than the IL-23p19 expression. Using the relation between the stimulation indexes of the IL-23p19 and IL-1-beta cytokines unexpectedly results in a predictive powerful tool.

It is clear of course that calculating the relation between the stimulation indexes of the IL-23p19 and IL-1-beta cytokines can be done in several ways:

In general, the equation used is R=Stimulation index IL-23p19/Stimulation index IL-1-beta, wherein the stimulation index can be either calculated as being level of the cytokine after stimulation/level of cytokine before stimulation, or vice versa.

Preferably the equation used is:

$$R 1 = \frac{\frac{Blood mRNA levels of IL \text{-} 23 p 19 after stimulation}{Blood mRNA levels of IL \text{-} 23 p 19 before stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index A ) \\ ( Stimulation index B )\end{matrix}$$

but rearrangements of the equation R1 will obviously also correlate to the MS disease status and can be calculated in any other way e.g. as follows:

$$R 2 = \frac{\frac{Blood mRNA levels of IL \text{-} 1 \text{-} beta after stimulation}{Blood mRNA levels of IL \text{-} 1 \text{-} beta before stimulation}}{\frac{Blood mRNA levels of IL \text{-} 23 p 19 after stimulation}{Blood mRNA levels of IL \text{-} 23 p 19 before stimulation}} \begin{matrix}( Stimulation index B ) \\ ( Stimulation index A )\end{matrix}$$

$$or \text{:}$$

$$R 3 = \frac{\frac{Blood mRNA levels of IL \text{-} 23 p 19 after stimulation}{Blood mRNA levels of IL \text{-} 23 p 19 before stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index {A}^{- 1} ) \\ ( Stimulation index {B}^{- 1} )\end{matrix}$$

$$or \text{:}$$

$$R 4 = \frac{\frac{Blood mRNA levels of IL \text{-} 1 \text{-} beta before stimulation}{Blood mRNA levels of IL \text{-} 1 \text{-} beta after stimulation}}{\frac{Blood mRNA levels of IL \text{-} 23 p 19 before stimulation}{Blood mRNA levels of IL \text{-} 23 p 19 after stimulation}} \begin{matrix}( Stimulation index {B}^{- 1} ) \\ ( Stimulation index {A}^{- 1} )\end{matrix}$$

$$or$$

$$R 5 = \frac{\frac{Blood mRNA levels of IL \text{-} 1 \text{-} beta after stimulation}{Blood mRNA levels of IL \text{-} 1 \text{-} beta before stimulation}}{\frac{Blood mRNA levels of IL \text{-} 23 p 19 after stimulation}{Blood mRNA levels of IL \text{-} 23 p 19 before stimulation}} \begin{matrix}( Stimulation index B ) \\ ( Stimulation index {A}^{- 1} )\end{matrix}$$

$$or$$

$$R 6 = \frac{\frac{Blood mRNA levels of IL \text{-} 23 p 19 before stimulation}{Blood mRNA levels of IL \text{-} 23 p 19 after stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index {A}^{- 1} ) \\ ( Stimulation index B )\end{matrix}$$

$$or$$

$$R 7 = \frac{\frac{Blood mRNA levels of IL \text{-} 1 \text{-} beta before stimulation}{Blood mRNA levels of IL \text{-} 1 \text{-} beta after stimulation}}{\frac{Blood mRNA levels of IL \text{-} 23 p 19 after stimulation}{Blood mRNA levels of IL \text{-} 23 p 19 before stimulation}} \begin{matrix}( Stimulation index {B}^{- 1} ) \\ ( Stimulation index A )\end{matrix}$$

$$or$$

$$R 8 = \frac{\frac{Blood mRNA levels of IL \text{-} 1 \text{-} beta after stimulation}{Blood mRNA levels of IL \text{-} 1 \text{-} beta before stimulation}}{\frac{Blood mRNA levels of IL \text{-} 23 p 19 before stimulation}{Blood mRNA levels of IL \text{-} 23 p 19 after stimulation}} \begin{matrix}( Stimulation index B ) \\ ( Stimulation index {A}^{- 1} )\end{matrix}$$

without losing the correlation between active and non-active MS status. Values of equations R2, R3, R5 and R8 will be decreased in active state MS vs. non-active state MS, while values of equations R1, R4, R6 and R7 will typically be increased in active state MS vs. non-active state MS.

In the examples below and the embodiments described herein, the equation R1 is used, but it will be clear that in essence any other equation using the stimulation indexes of IL-1-beta and IL-23p19 will be correlated to the MS activity status. In all examples and embodiments defined herein, the equation R1 can thus be replaced by any other equation or rearrangement thereof. Non-limiting examples of such alternative equations are the ones described above and denominated as R2-R8, or rearrangements thereof.

In alternative embodiments of the methods and kits of the invention, the markers IL23p19 and IL-1-beta can be replaced by IL-17 or IL-6 and IL-1-beta respectively. Other putative important markers are ROR-γt, IL-21, IL-22, FOXp3, IL-10, TGF-β, IFN-g, IL-8 or Kruppel-like factor 4 (gut) (KLF4).

Human beta interferon has been demonstrated to modulate many of the biological processes underlying the pathology of MS (cf. e.g. Goodin D S 2005, The Int. MS Journal, vol. 12:96-108). Beta interferon belongs to a large family of secreted proteins, collectively referred to as the interferons, which are involved in an organism's defense against viral infections, in the regulation of cell growth and in the modulation of immune responses.

There are two basic types of interferon: type I interferons (mainly a and b) are secreted principally by leucocytes (a or alpha) and fibroblasts (b or beta) and are induced directly in response to a viral infection; type II interferons (g or gamma) are synthesized by T-lymphocytes or natural killer cells following the detection of infected cells by antigen presentation. Human beta interferon is a naturally occurring glycoprotein, 166 amino acids in length and with a molecular weight of 22.5 kDa. It is 30-40% homologous with the multi-gene alpha interferon family. Moreover, it is encoded on chromosome 9 without introns, similar to the principal form of alpha interferon. Both type I interferons (a and b) bind to the same two-subunit cell-surface receptor (interferon a receptor 1 and 2; also called IFNAR1 and IFNAR2), which is encoded on chromosome 21. In addition, both activate the same Janus kinase/signal transducer and activator of the transcription (JAK/STAT) signaling pathway, which leads ultimately to the binding of interferon-stimulated gene factor 3 (ISGF3) to the interferon-stimulated response element (ISRE). The ISRE is a short consensus DNA sequence (approximately 10-12 bases in length), which is a component of several nuclear genes. Other molecules can also bind to the ISRE, such as members of the family of interferon regulatory factors (IRFs), some of which are induced by beta interferon. Following the binding of ISGF3 to the ISRE, the transcription of these ISRE-containing genes, which would otherwise be expressed at low or very low levels, is activated. These beta interferon-induced gene products include neopterin, 2′-5′ oligoadenylate synthase (2.5 OAS), the Mx family of GTPases (including MxA and MxB), b2 microglobulin, dsRNA-dependent protein kinase (PKR), IRF-1, IRF-2, IRF-7, and major histocompatibility complex (MHC) class I molecules. Importantly, despite the binding of alpha and beta interferon to the same cell-surface receptor, the downstream expression pattern of these ISRE-containing genes is not identical following exposure to these two molecules.

Gamma interferon is not homologous to either alpha or beta interferon. Located on chromosome 12 with three introns, it binds to a different two-subunit cell-surface receptor (interferon g receptor 1 and 2; also called IFNGR1 and IFNGR2), which is encoded on chromosome 21, and it activates a different, but related, JAK/STAT signaling pathway.

The present method may be applied to any individual suffering from a disease which can be treated with an immunomodulator such as a type I interferon, including for instance multiple sclerosis, chronic hepatitis C (HCV), or chronic hepatitis B (HBV). The terms “individual” or “patient” or “subject” are used herein as synonym and preferably refer to a human suffering from a disease which can be treated with a type I interferon. Such individual may for instance comprise a patient suffering from multiple sclerosis, (chronic) hepatitis C (HCV) and/or B (HBV), etc.

The term “immunomodulator” as used herein generally refers to type-1 interferon (type I IFN), including IFN-alpha and IFN-beta, and the commercial type I IFN products known as Avonex® (from Biogen), Rebif® (from Serono), Betaseron® (from Berlex), Betaferon®, (from Schering) or other agents having similar effects or use in MS such as Glatiramer Acetate (Copaxone®); synthetic polypeptides with a structure resembling myelin, Natalizumab (Tysabri®); anti-CD52 such as Alemtuzumab® (CAMPATH), anti-CD25, such as Daclizumab®; agents acting on the sphingosine receptors such as FTY720 (Fingolimod®); agents having an sequestering effect on lymphocytes; agents depleting T-lymphocytes, such as Cladribine®, or Teriflunomide®; Th2 response inducing agents such as BG-12 from Biogen, which is Fumarate, and the like.

In a preferred embodiment, the type I interferon according to the invention may comprise an IFN-beta or an IFN-alpha interferon, depending on the disease which affects the individual to be monitored. In one embodiment, the treatment with a type I interferon involves a treatment of multiple sclerosis with an IFN-beta. Preferably, said treatment with an IFN-beta comprises a treatment with an IFN-beta-1a or an IFN-beta-1b.

Examples of pharmaceutical preparations comprising IFN-beta-1a may comprise the commercially available Avonex® (Biogen) or Rebif® (Serono) drugs. They are produced in a Chinese hamster ovary (CHO) cell line. Similar to native human beta interferon, IFNb-1a is a glycoprotein that has the complete 166 amino acid sequence of the native human molecule.

Examples of pharmaceutical preparations comprising IFN-beta-1b may comprise Betaseron® (Berlex) or Betaferon® (Schering), produced in an Escherichia coli cell line. As bacteria do not glycosylate proteins, IFNb-1b has no sugar molecules attached.

In another preferred embodiment, the treatment with an immunomodulator such as a type I interferon involves a treatment of chronic hepatitis C (HCV) with an IFN-alpha. Said treatment with an IFN-alpha may comprise a treatment with an IFN-alpha-2a or an IFN-alpha-2b. Examples of pharmaceutical preparations comprising IFN-alpha-2a may comprise Pegasys® and Roferon®. Examples of pharmaceutical preparations comprising IFN-alpha-2b may comprise Intron A® and Pegintron®.

The term “blood sample” applied in the present method generally refers to a “whole blood sample”. The term “whole blood” as used herein refers to blood as it is collected by venous sampling, i.e. containing white and red cells, platelets, and plasma.

The mRNA levels of the cytokines can be determined using any known method in the art. Examples are: Polymerase Chain Reaction (PCR), Real-Time quantitative PCR (RT-qPCR), End-Point PCR, digital PCR (dPCR), RNA or cDNA hybridization techniques, microarrays, RNA-in-situ hybridization (RISH), Northern-Blotting, digital analysis of gene expression (DAGE), sequence-analysis based expression analysis, Supported oligonucleotide detection, Pyrosequencing, Polony Cyclic Sequencing by Synthesis, Simultaneous Bi-directional Sequencing, Single-molecule sequencing, Single molecule real time sequencing, True Single Molecule Sequencing, Hybridization-Assisted Nanopore Sequencing or Sequencing by synthesis.

In a preferred embodiment, the cytokine mRNA levels are determined by real-time quantitative polymerase chain reaction (qPCR, qc-PCR, RT-PCR). As used herein, “Real-time quantitative rt-PCR” relates to a method that monitors the degradation of a dual-labeled fluorescent probe in real time concomitant with PCR amplification. Input target RNA levels are correlated with the time (measured in PCR cycles) at which the reporter fluorescent emission increases beyond a threshold level. In example 1, a real-time quantitative polymerase chain reaction (qc-PCR) as can be applied in accordance with the present invention is illustrated.

The present method thus comprises obtaining or providing two blood samples from a subject, one sample before the subject is treated with the immunomodulating agent and one sample after the subject is treated with the immunomodulating agent. The blood samples are not further treated and are analyzed as such. In-between the taking of both blood samples, the subject is submitted to a treatment with the immunomodulating agent during a suitable period of time which may vary from 1 to 24 hours, depending on the immunomodulating agent that is administrated to the patient; and which for instance may be about 2, 3, 4, 5, 6, 7 or 8 hours for IFN-beta-1a; 2, 3, 4, 5, 6, 7, 8, 9, 10, 11, 12 hours for IFN-beta-1b.

Alternatively, the immunomodulator is not administered to the subject under treatment, but is added in vitro to the sample or a portion thereof obtained from the subject. In this case, a single sample of the subject can be divided in two portions: one portion to which the immunomodulator is added in vitro and one portion to which no immunomodulator is added. The stimulation index of the two cytokines as defined in the present invention can then be calculated based on the differential mRNA levels of said cytokines between both samples, i.e. with and without immunomodulator added.

In a preferred embodiment, before mRNA determination, all blood samples (i.e. the blood samples obtained from a subject before and after treatment with the immunomodulator or the blood samples incubated or not with an immunomodulator in vitro) are all treated with a stabilizing agent. In an embodiment, the stabilizing agent is an inhibitor of cellular RNA degradation and/or gene induction. For example, said inhibitor of cellular RNA degradation and/or gene induction is that as found in a PAXgene™ Blood RNA Tube or alternatively in a Tempus™ Blood RNA Tube. For example, a quaternary amine surfactant may be used as a stabilizing agent. Suitable quaternary amine surfactants, able to stabilize RNA from biological samples, are described in U.S. Pat. No. 5,985,572, WO94/18156 and WO02/00599. One example of a quaternary amine which can be used in the method of the present invention is tetradecyltrimethyl-ammonium oxalate. (U.S. Pat. No 5,985,572). Alternatively, said cationic detergent may be Catrimox-14™ (U.S. Pat. No 5,010,183).

The method of the invention can comprise the steps of:
a) providing a first and a second blood sample (or a first and second portion of a blood sample) of a subject, wherein the first sample is taken prior to the treatment with an immunomodulator and the second sample is taken after the treatment with the immunomodulator,
b) adding to said first and second blood sample a stabilizing agent,
c) determining the mRNA levels of the IL-1-beta and IL-23p19 cytokines in said first and second stabilized blood samples;
d) comparing the mRNA levels of the IL-1-beta and IL-23p19 cytokines determined in step b) and c), and
e) evaluating the active status of the MS in said individual under treatment, preferably by calculating the following equation:

$$R 1 = \frac{\frac{Blood mRNA levels of IL \text{-} 23 p 19 after stimulation}{Blood mRNA levels of IL \text{-} 23 p 19 before stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index A ) \\ ( Stimulation index B )\end{matrix}$$

In a preferred embodiment, the method of the present invention may use a blood collecting vessel and a container in which a stabilizing agent is present. Preferably, the inside of said blood collecting vessel and the inside of said container are connected, and a physical barrier temporarily blocks said connection.

The method of the invention can comprise the steps of:
a) providing a first and a second blood sample (or a first and second portion of a blood sample) of a subject, wherein the first sample is taken prior to the treatment with an immunomodulator and the second sample is taken after the treatment with the immunomodulator,
b) adding said first and second blood sample in a separate device comprising: (i) a collection vessel, (ii) a container in which a stabilizing agent is present, (iii) a connection between the inside of said vessel and the inside of said container, and (iv) a physical barrier that temporarily blocks said connection.
c) adding to said first and second blood sample the stabilizing agent by removing said physical barrier,
d) determining the mRNA levels of the IL-1-beta and IL-23p19 cytokines in said first and second stabilized blood samples;
e) comparing the mRNA levels of the IL-1-beta and IL-23p19 cytokines determined in step c) and d), and
f) evaluating the active status of the MS in said individual under treatment, preferably by calculating the following equation:

$$R 1 = \frac{\frac{Blood mRNA levels of IL \text{-} 23 p 19 after stimulation}{Blood mRNA levels of IL \text{-} 23 p 19 before stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index A ) \\ ( Stimulation index B )\end{matrix}$$

Alternatively, the method of the present invention may use a blood collecting vessel and a container in which a stabilizing agent is present as described above, wherein either an amount of immunomodulator is present in the vessel (i) used for collecting the second blood sample, or wherein said blood collecting vessel additionally comprises a container (v) comprising an amount of immunomodulator, connected with vessel (i) by a connection (vi), said connection being temporarily blocked by a physical barrier (vii).

The method of the invention comprises the steps of:
a) providing a first and a second blood sample (or a first and second portion of a blood sample) of a subject, preferably before the subject was treated with an immunomodulator,
b) adding said first and second blood sample in a separate device comprising: (i) a collection vessel, (ii) a container in which a stabilizing agent is present, (iii) a connection between the inside of said vessel and the inside of said container, and (iv) a physical barrier that temporarily blocks said connection, additionally comprising a container (v) comprising an amount of immunomodulator, connected with vessel (i) by a connection (vi), said connection being temporarily blocked by a physical barrier (vii).
c) contacting only one of the samples or portion of the samples with an immunomodulator e.g. by removing a temporary physical barrier (vii) separating the sample vessel (i) and the container (v) with the immunomodulator,
d) incubate the samples or portion of the samples in the separate device for a suitable time period
e) adding to said first and second (portion of the) blood sample the stabilizing agent e.g. by removing a temporary physical barrier (iv) separating the sample vessel (i) and the container (ii) with the stabilizing agent,
f) determining the mRNA levels of the IL-1-beta and IL-23p19 cytokines in said first and second stabilized (portions of) blood samples;
g) comparing the mRNA levels of the IL-1-beta and IL-23p19 cytokines determined in step d) and e), and
h) evaluating the active status of the MS in said individual under treatment, preferably by calculating the following equation:

$$R 1 = \frac{\frac{Blood mRNA levels of IL \text{-} 23 p 19 after stimulation}{Blood mRNA levels of IL \text{-} 23 p 19 before stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index A ) \\ ( Stimulation index B )\end{matrix}$$

The first and second (portions of) blood samples are preferably stabilized with stabilizing agent, as fast as possible after blood collection or after the ex vivo incubation.

In any one of the embodiments listed or defined herein, the immunomodulatory agent can also be added in vitro, i.e. after the sample of e.g. blood has been taken from the subject under investigation. One part of the blood sample is then immunostimulated with the modulatory agent and the resulting RNA expression pattern resulting from the stimulation is determined subsequently by fixing the RNA with an RNA stabilizing agent, followed by RNA analysis as indicated for the other embodiments. The remaining part of the blood sample is also analysed for its RNA expression pattern in the same manner, safe from the addition of the immunomodulatory agent. The two expression patterns can then be compared to give an indication of the active state of the MS in the subject. In this embodiment, the immunostimulatory response of the subject is not tested in vivo, but is tested in vitro in a test tube comprising the whole blood sample taken from the subject.

In another preferred embodiment, the method involves incubating a blood sample in the presence of an immunomodulator in an incubator, preferably at a temperature of about 37° C. Preferably, the method is performed in the absence of controlling the air composition during incubation. In accordance with the invention, as incubator an oven can be used that is working under ambient atmospheric conditions, i.e. without any regulation of the amounts of CO₂ and H₂O present in the oven. In said oven, the sample is preferably maintained at 37° C. However, a cell culture incubator, wherein atmospheric conditions and the concentration of e.g. CO₂ are controlled, is not required for carrying out the incubation step of the present method.

In a particularly preferred embodiment, the present method for assessing the active state of MS in a subject comprises the steps of:
a) providing a first and a second (portion of a) blood sample of said individual, preferably prior to in vivo treatment of said individual with an immunomodulator,
b) adding in vitro to said second (portion of a) blood sample, a suitable amount of said immunomodulator;
c) incubating the second (portion of a) sample of step b) in vitro for a suitable period of time;
d) determining IL-1-beta and IL-23p19 mRNA levels in the first (portion of a) blood sample of step a);
e) determining IL-1-beta and IL-23p19 mRNA levels in the incubated second (portion of a) blood sample of step c);
f) comparing IL-1-beta and IL-23p19 mRNA levels determined in step d) and e), and
g) evaluating the active state of MS in said subject based on the in vitro results obtained in step f) by calculating the following equation:

$$R 1 = \frac{\frac{Blood mRNA levels of IL \text{-} 23 p 19 after stimulation}{Blood mRNA levels of IL \text{-} 23 p 19 before stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index A ) \\ ( Stimulation index B )\end{matrix}$$

In a particularly preferred embodiment, the present method for assessing the active state of MS in a subject comprises the steps of:
a) providing a first and a second (portion of a) blood sample of said individual, preferably prior to in vivo treatment of said individual with an immunomodulator,
b) adding in vitro to said second (portion of a) blood sample, a suitable amount of said immunomodulator;
c) incubating the second (portion of a) sample of step b) in vitro for a suitable period of time;
d) determining IL-1-beta and IL-23p19 mRNA levels in the first (portion of a) blood sample of step a);
e) determining IL-1-beta and IL-23p19 mRNA levels in the incubated second (portion of a) blood sample of step c);
f) comparing IL-1-beta and IL-23p19 mRNA levels determined in step d) and e), and
g) evaluating the active state of MS in said subject based on the in vitro results obtained in step f) by calculating the following equation:

$$R 1 = \frac{\frac{Blood mRNA levels of IL \text{-} 23 p 19 after stimulation}{Blood mRNA levels of IL \text{-} 23 p 19 before stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index A ) \\ ( Stimulation index B )\end{matrix}$$

The present method thus comprises obtaining one or two blood samples from an individual before the individual is treated with the immunomodulator. One blood sample or a part of the blood sample is not further treated and analyzed as such. The second blood sample or the other part of the blood sample is incubated and stimulated in vitro with a suitable amount of an immunomodulator during a suitable period of time which may vary from 4 to 12 hours, depending on the IFN that is added to the blood; and which for instance may be about 4, 5, 6, 7 or 8 hours for IFNβ-1a; 4, 5, 6, 7, 8, 9, 10, 11, 12 hours for IFNβ-1b.

In a preferred embodiment, after incubation and before mRNA determination, the samples are treated with a stabilizing agent. In an embodiment, the stabilizing agent is an inhibitor of cellular RNA degradation and/or gene induction. For example, said inhibitor of cellular RNA degradation and/or gene induction is that as found in a PAXgene™ Blood RNA Tube. For example, a quaternary amine surfactant may be used as a stabilizing agent. Suitable quaternary amine surfactants, able to stabilize RNA from biological samples, are described in U.S. Pat. No. 5,985,572, WO94/18156 and WO02/00599. One example of a quaternary amine which can be used in the method of the present invention is tetradecyltrimethyl-ammonium oxalate. (U.S. Pat. No 5,985,572). Alternatively, said cationic detergent may be Catrimox-14™ (U.S. Pat. No 5,010,183).

In an embodiment, the method of the present invention may use a vessel comprising a type I interferon and a container in which a stabilizing agent is present. Preferably, the inside of said vessel and the inside of said container are connected, and a physical barrier temporarily blocks said connection.

The method of the invention can comprise the steps of:
a) providing a first and a second blood sample (or a first and second portion of a blood sample) of an individual, preferably before the individual was treated with an immunomodulator,
b) adding only said second blood sample (or said second blood portion) in a vessel comprising: (i) a suitable amount of immunomodulator present inside said vessel, (ii) a container in which a stabilizing agent is present, (iii) a connection between the inside of said vessel and the inside of said container, and (iv) a physical barrier that temporarily blocks said connection.
c) incubating said second blood sample (or second blood portion) in vitro for a suitable period of time;
d) adding to said second blood sample (or second blood portion) the stabilizing agent by removing said physical barrier,
e) determining IL-1-beta and IL-23p19 mRNA levels in said incubated and stabilized blood sample (or second blood portion);
f) adding to said first blood sample (or first blood portion) a stabilizing agent and determining IL-1-beta and IL-23p19 mRNA levels in a said first blood sample (or first blood portion) from said patient,
g) comparing IL-1-beta and IL-23p19 mRNA levels determined in step e) and f), and
h) evaluating the active state of MS in the individual by calculating the equation:

$$R 1 = \frac{\frac{Blood mRNA levels of IL \text{-} 23 p 19 after stimulation}{Blood mRNA levels of IL \text{-} 23 p 19 before stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index A ) \\ ( Stimulation index B )\end{matrix}$$

In an embodiment, said first blood sample (or first blood portion) is also incubated, similarly to said second sample (or blood portion), in a vessel as described above but free of immunomodulator. This first blood sample is also stabilized with stabilizing agent, as fast as possible after blood collection, or alternatively, after incubation as done for the tube containing the immunomodulator. This can again be done by perforating or removing the temporary physical barrier (iv) between the sample vessel (i) and the container (ii) comprising the stabilizing agent. In a preferred embodiment, the immunomodulator is present in the sample vessel prior to the addition of the second (portion of the) sample.

In an alternative embodiment, said immunomodulator is present in an additional container (v), in connection with said sample vessel, wherein the connection (vi) is temporary blocked by a physical barrier (vii). In this embodiment, two identical vessels can be used for the first and second (portion of) the blood sample, wherein for the first (portion of the) sample, the physical barrier between the immunomodulator container and the sample vessel is left intact, and wherein for the second (portion of the) sample, said barrier is perforated, thereby only bringing the second (portion of the) sample in contact with the immunomodulator.

The method comprises the determination of IL-1-beta and IL-23p19 mRNA levels in the first blood sample of said individual prior to treatment and in the second blood sample that has been incubated with the immunomodulator. The mRNA level of IL-1-beta and IL-23p19 is compared in both samples and based on the results thereof, the active status of MS in said individual is evaluated.

In a further embodiment, the method comprises the step of calculating a stimulation index, corresponding to the IL-1-beta and IL-23p19 mRNA levels after in vivo or in vitro stimulation divided by the IL-1-beta and IL-23p19 mRNA levels before said stimulation.

In another embodiment, the present invention therefore also provides a method for monitoring the in vivo response of an individual to a treatment with an immunomodulator, comprising incubating a blood sample of said individual in vitro with a suitable amount of said immunomodulator for a suitable period of time, and determining mRNA levels of the gene encoding IL-1-beta and IL-23p19 in said blood sample. The method can be applied using the steps, conditions, amounts, examples of incubation times and conditions as described above.

In yet another embodiment the invention also provides a method for identifying an individual as a responder or non responder to a treatment with an immunomodulator, comprising incubating a blood sample of said individual in vitro with a suitable amount of said immunomodulator for a suitable period of time, and determining mRNA levels of the gene encoding IL-1-beta and IL-23p19 in said blood sample. The method can be applied using the steps, conditions, amounts, examples and incubation times and conditions as described above.

The invention further provides a method for the in vitro induction of IL-1-beta and IL-23p19 mRNA expression by an immunomodulator in mammalian whole blood. The method comprises incubating said whole blood in the presence of a suitable amount of an immunomodulator for a suitable period of time. Suitable amounts, examples and incubation times and conditions are similar as those given above.

The invention further provides a method for adjusting an IFN-therapy in a patient, comprising the step of identifying an individual as a responder or non responder to a treatment with a type I interferon using the method of the present invention, and adjusting said IFN-therapy when the patient is a non-responder. In an embodiment, said adjusting step comprises discontinuing the therapy. In another embodiment, said adjusting step comprises using a less immunogenic IFN or glatiramer acetate. Preferably, the identification step is performed at least twice before adjusting said IFN-therapy. Preferably, the at least two successive identification steps are separated by 3 to 6 months. The method can be applied using the steps, conditions, amounts and examples of preferred type I IFN and incubation times and conditions or as described above.

Also provided are kits for use in practicing the subject methods. The term “kit” as used herein refers to any combination of reagents or apparatus that can be used to perform a method of the invention.

A kit according to the invention typically comprises:
a) one or more vessel(s) suitable for accepting a blood sample,
b) a primer pair specific to the mRNA of the IL-23p19 gene which is suitable for the transcription of mRNA of said control gene into cDNA and the amplification of the latter, and a probe designed to anneal to an internal region of the produced cDNA,
c) a primer pair specific to the mRNA of the IL-1-beta gene which is suitable for the transcription of mRNA of said control gene into cDNA and the amplification of the latter, and a probe designed to anneal to an internal region of the produced cDNA,

wherein said vessel comprises: i) a vessel capable of accepting a blood sample, and optionally ii) a container in which a stabilizing agent is present, iii) a connection between the inside of said vessel (i) and the inside of said container (ii), and iv) a physical barrier that temporarily blocks said connection.

The kit according to the invention optionally additionally comprises:
d) a control primer pair specific to the mRNA of a control gene which is suitable for the transcription of mRNA of said control gene into cDNA and the amplification of the latter, and a control probe designed to anneal to an internal region of the produced control cDNA.

In any one of the embodiments listed or defined herein, the immunomodulatory agent can either be added in vitro, i.e. after the sample of e.g. blood has been taken from the subject under investigation. One part of the blood sample is then immunostimulated with the modulatory agent and the resulting RNA expression pattern resulting from the stimulation is determined subsequently by fixing the RNA with an RNA stabilizing agent, followed by RNA analysis as indicated for the other embodiments. The remaining part of the blood sample is also analysed for its RNA expression pattern in the same manner, safe from the addition of the immunomodulatory agent. The two expression patterns can then be compared to give an indication of the active state of the MS in the subject.

A typical kit according to the invention can thus comprise:
a) one or more vessel suitable for accepting a blood sample,
b) a primer pair specific to the mRNA of the IL-1-beta gene and a primer pair specific to the mRNA of the IL-23p19 gene, and
c) a probe designed to anneal to an internal region of the produced IL-1-beta and IL-23p19 cDNA,

wherein said vessel (i) comprises: a) an immunomodulator present inside said vessel, optionally present in a container (v) separated from said vessel by a physical barrier (vii) temporarily blocking the connection (vi), b) a container (ii) in which an RNA-stabilizing agent is present, c) a connection between the inside of said vessel (i) and the inside of said container (ii), d) a physical barrier (iv) that temporarily blocks said connection (iii).

In use, any one of the physical barrier(s) in the kits or methods of the invention may be opened by the application of physical force to said vessel. Said force may transmit an opening means to said physical barrier. Examples of such physical barriers include rotary valve, aperture valve, slit valve, diaphragm valve, ball valve, flap valve. Alternatively, said force may irreversibly open said physical barrier. Other examples of such physical barriers include a plug which is forced out of position, a barrier which shatters upon the application of force. In an embodiment, the inside of said container and the inside of said vessel are connected, and the flow of stabilizing agent from the container to the vessel is prevented by the surface tension of the stabilizing agent in combination with the aperture size of the connection. According to this aspect, at an appropriate time an application of force which transmits to the stabilizing agent, forces the stabilizing agent from the container into the vessel. The force may be applied, for example, by squeezing, continually inverting, and agitating.

The immunomodulator such as a type I interferon can be provided in said vessel in a liquid or lyophilized form, not immobilized. The immunomodulator can also be immobilized on part or all of the inside surface of said vessel. The inside wall of the vessel may be lined with a suitable coating enabling the immunomodulator to be attached. In another embodiment, said immunomodulator is immobilized on a solid support. The solid support may be attached to the inside of the vessel. Alternatively, the solid support may be free of the inside of the vessel. Examples of solid supports include, but are not limited to, chromatography matrix, magnetic beads.

The vessel may be sealed with resealing means such as a screw-cap, push-on cap, a flip-cap. Said vessel may comprise one or more openings. In a particular embodiment, the vessel as described above comprises one or more areas suitable for puncture by a syringe needle, such as a re-sealable septum. The vessel may comprise a fitting suitable for receiving a syringe or a syringe needle and transmitting the contents therein to the interior of said vessel. Suitable vessel may further comprise cannular suitable for withdrawing bodily fluids.

Suitable vessel may further comprise a valve which is capable of minimizing the flow of gas/liquid from vessel, and allowing the flow of biological sample into the vessel. Suitable vessel may further comprise a means through which displaced gas may be expelled. Said means are known the art and include valves, non-drip holes, vents, clothed-vents, expandable vessel walls, use of negative pressure within said vessel. Said vessel may further be held under negative pressure. The negative pressure may be utilized to relieve the pressure build-up upon introduction of whole blood into said sealed vessel. Alternatively, or in addition, the negative pressure may be at a predetermined level and may be utilized so as to allow the introduction of a fixed volume of whole blood. Suitable vessel may comprise an indication for dispensing a known volume of stabilizing agent therein.

In an embodiment, the stabilizing agent is an inhibitor of cellular RNA degradation and/or gene induction. For example, said inhibitor of cellular RNA degradation and/or gene induction is that as found in a PAXgene™ Blood RNA Tube or alternatively in a Tempus™ Blood RNA Tube. For example, a quaternary amine surfactant may be used as a stabilizing agent. Suitable quaternary amine surfactants, able to stabilize RNA from biological samples, are described in U.S. Pat. No. 5,985,572, WO94/18156 and WO02/00599. One example of a quaternary amine which can be used in the method and kits of the present invention is tetradecyltrimethyl-ammonium oxalate. (U.S. Pat. No 5,985,572). Alternatively, said cationic detergent may be Catrimox-14™ (U.S. Pat. No 5,010,183).

Preferably, the primer pair specific to the mRNA of the IL-23p19 gene comprises oligonucleotide sequences represented by SEQ ID NO: 1 and SEQ ID NO: 2. Preferably, the probe designed to anneal to an internal region of the produced comprises an oligonucleotide sequence represented by SEQ ID NO: 3.

Preferably, the primer pair specific to the mRNA of the IL-1-beta gene comprises oligonucleotide sequences represented by SEQ ID NO: 4 and SEQ ID NO: 5. Preferably, the probe designed to anneal to an internal region of the produced comprises an oligonucleotide sequence represented by SEQ ID NO: 6.

In an embodiment, said control gene is selected from the group comprising mRNAs for certain ribosomal proteins such as RPLP0 (ribosomal protein, large, P0), glyceraldehyde-3-phosphate dehydrogenase mRNA, beta actin mRNA, MHC I (major histocompatibility complex I) mRNA, cyclophilin mRNA, 28S or 18S rRNAs (ribosomal RNAs). In a preferred embodiment, said control gene is the Human Acidic Ribosomal Phosphoprotein P0 (RPLP0 gene). In a preferred embodiment, said primer pair specific to the mRNA of said control gene comprises oligonucleotide sequences represented by SEQ ID NO: 7 and SEQ ID NO: 8 or represented by SEQ ID NO: 9 and SEQ ID NO: 10. Preferably the probe designed to anneal to an internal region of the produced control cDNA, comprises an oligonucleotide sequence represented by SEQ ID NO: 11 or by SEQ ID NO: 12.

The kit can further comprise additional components for carrying out the method of the invention, such as RNA extraction solutions, purification column and buffers and the like. The kit of the invention can further include any additional reagents, reporter molecules, buffers, excipients, containers and/or devices as required described herein or known in the art, to practice a method of the invention.

The various components of the kit may be present in separate containers or certain compatible components may be pre-combined into a single container, as desired. In addition to the above components, the kits may further include instructions for practicing the present invention. These instructions may be present in the kits in a variety of forms, one or more of which may be present in the kit.

One form in which these instructions may be present is as printed information on a suitable medium or substrate, e. g., a piece or pieces of paper on which the information is printed, in the packaging of the kit, in a package insert, etc. Yet another means would be a computer readable medium, e. g., diskette, CD, etc., on which the information has been recorded. Any convenient means may be present in the kits.

The following examples are intended to illustrate and to substantiate the present invention are not to be seen as limiting the invention.

### EXAMPLES

The invention is illustrated by the following non-limiting examples

### Example 1

### Correlating Cytokine Levels with Activity of MS in Patients

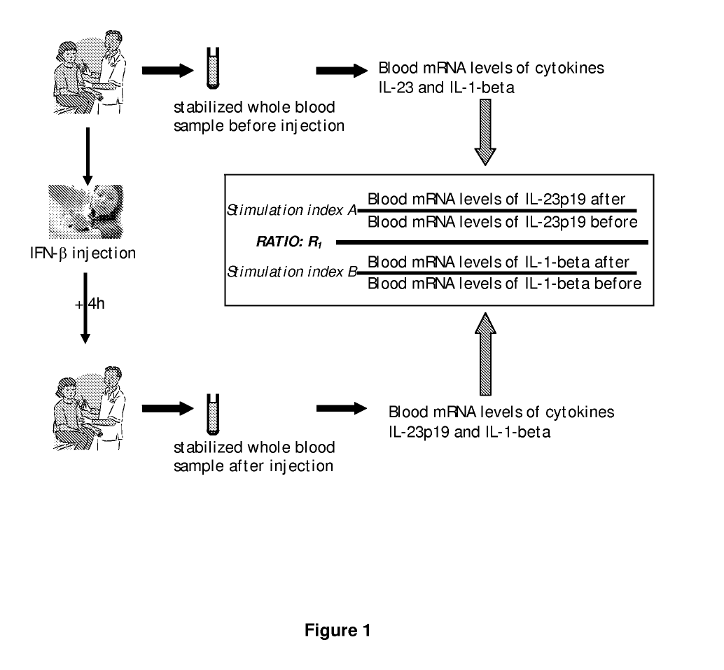

The procedure is schematised in FIG. 1)

Patients and Methods

51 MS patients that where under type I IFN treatment were observed in this study. Patients consent to the study was obtained. The patients suffered from MS in several different disease states. In vivo injection of IFN was performed as follows: the day of the experiment, administered IFNs comprised: IFNβ-1a (Rebif® 44 or 22 μg subcutaneously) or Avonex® (30 μg intramuscularly) or IFNβ-1b (Betaferon® 62.5 μg or 250 μg subcutaneously).

Clinical Classification of Patients as Being Active or Non-Active

Independent from the analysis of the cytokine levels as described below, the patients were clinically classified as having active MS when at least one of the following criteria were fulfilled:

1) an increase in the EDSS value during the last 6 months period,

2) increased demyelination (lesions or plaques) during the last 6 months using magnetic resonance imaging of the brain and spine with Gadolinium as a contrast agent,

3) patients suffered one attack while being treated or 2 attacks without being treated during the last 6 months.

In total, 51 patients were tested, of which initially 30 were classified as having active MS, and 21 were classified as having non-active MS. During the course of the analysis, three patients developed from a non-active MS status to an active MS status and from these patients, samples were analysed of both the active and non-active period (cf. Table 1).

Sample Preparation

First blood samples were taken before the treatment and mRNA extraction is carried out as previously described (Stordeur et al, 2002; J. Immunol. Methods 259:55-64+262:229 (erratum) and Stordeur et al, 2003; J. Immunol. Methods 276:69-77). Briefly, stimulation is stopped by adding the reagent contained in PAXgene™ tubes (Qiagen, Westburg, The Netherlands). This reagent induced total cell lysis and mRNA stabilization by nucleic acids precipitation. The nucleic acids pellet is then dissolved in the lysis buffer contained in the MagNA Pure™ mRNA extraction kit (Roche Applied Science, Roche Diagnostics Belgium, Vilvoorde, Belgium). mRNA is extracted from 300 μl of this solution, using this kit on the MagNA Pure™ instrument (Roche Applied Science) following manufacturer's instructions (“mRNA I cells” Roche's protocol). The second blood samples were taken approximately 4 hours after the administration of the type I IFN and mRNA extraction was done as for the first blood sample.

Q-PCR Analysis

Reverse transcription and real-time PCR was carried out as previously described (Stordeur et al, 2002; J. Immunol. Methods 259:55-64+262:229 (erratum) and Stordeur et al, 2003; J. Immunol. Methods 276:69-77), in one step on a LightCycler instrument, following the standard procedure described in the “LightCycler—RNA Master Hybridization Probes” Kit (Roche Applied Science).

Oligonucleotides sequence and final concentration for the IL-23p19 mRNA are as follows: TACTGGGCCTCAGCCAACT (SEQ ID NO: 1) at 900 nM for the forward primer, GAAGGATTTTGAAGCGGAGAA (SEQ ID NO: 2) at 900 nM for the reverse primer, and CCTCAGTCCCAGCCAGCCATG (SEQ ID NO: 3) at 200 nM for the probe.

Oligonucleotides sequence and final concentration for the IL-1-beta mRNA are as follows: ACAGATGAAGTGCTCCTTCCA (SEQ ID NO: 4) at 600 nM for the forward primer, GTCGGAGATTCGTAGCTGGAT (SEQ ID NO: 5) at 900 nM for the reverse primer, and CTCTGCCCTCTGGATGGCGG (SEQ ID NO: 6) at 200 nM for the probe.

After an incubation period of 20 minutes at 61° C. to allow mRNA reverse transcription, and then an initial denaturation step at 95° C. for 30 s, temperature cycling is initiated. Each cycle consists of 95° C. for 0 (zero) second and 60° C. for 20 s, the fluorescence being read at the end of this second step. 45 cycles are performed, in total. For each sample, the mRNA copy number is calculated from a standard curve. The latter is constructed for each PCR run from serial dilutions of a purified DNA. mRNA levels are expressed in absolute copy numbers normalized against house keeping gene mRNA (IL-23p19 or IL-1-beta mRNA copies per million of reference gene mRNA copies). RPLPO (Human Acidic Ribosomal Phosphoprotein P0) was used as house keeping gene, with the following oligonucleotide sequence and final concentration: TGTCTGTCTGCAGATTGGCTAC (SEQ ID NO: 7) at 300 nM for the forward primer, AGATGGATCAGCCAAGAAGG (SEQ ID NO: 8) at 600 nM for the reverse primer, and CGGATTACACCTTCCCACTTGCTGA (SEQ ID NO: 9) at 200 nM for the probe, or alternatively, CCTTTGGGCTGGTCAT (SEQ ID NO: 10) at 300 nM for the forward primer, GCACTTCAGGGTTGTAG (SEQ ID NO: 11) at 900 nM for the reverse primer, and CCAGCAGGTGTTCGACAATGGC (SEQ ID NO: 12) at 200 nM for the probe.

ROC-Analysis

Using the Q-PCR values obtained as described above, the equation R1 was determined using the following formula:

$$R 1 = \frac{\frac{Blood mRNA levels of IL \text{-} 23 p 19 after stimulation}{Blood mRNA levels of IL \text{-} 23 p 19 before stimulation}}{\frac{Blood mRNA levels of IL \text{-} 1 \text{-} beta after stimulation}{Blood mRNA levels of IL \text{-} 1 \text{-} beta before stimulation}} \begin{matrix}( Stimulation index IL \text{-} 23 p 19 ) \\ ( Stimulation index IL \text{-} 1 \text{-} β )\end{matrix}$$

The R1-values of the patients pre-classified into active and non-active groups based on the clinical analysis indicated above were plotted on a ROC curve.

Results

IFN-b-treated multiple sclerosis (MS) patients were divided in two groups according to their disease status, i.e. clinically active or not, based on several parameters including EDSS score and MRI. The blood mRNA levels for the two cytokines IL-23p19 and IL-1-beta were evaluated, before and 4 hours after IFN-b injection. A stimulation index (mRNA level after vs. mRNA level before injection) was calculated for the two cytokine mRNAs, and these two indexes were used to calculate the ratio cytokine IL-23p19/cytokine IL-1-beta (Table 1).

TABLE 1

| | Disease status | | | |
|---|---|---|---|---|
| | A = active | IL-1β | IL-23p19 | |
| | NA = non | stimulation | stimulation | |
| Patient | active | index A | index B | R1 |
| AnAn | A | 1.16 | 4.02 | 3.47 |
| BeSe | A | 1.14 | 1.00 | 0.88 |
| BoBr | A | 0.92 | 2.41 | 2.62 |
| BuMa | A | 2.13 | 3.69 | 1.73 |
| ChBe | A | 1.61 | 2.39 | 1.48 |
| CoCi | A | 2.96 | 12.57 | 4.25 |
| DePh | A | 1.31 | 2.04 | 1.56 |
| HaFa | A | 2.34 | 3.94 | 1.68 |
| JuAm | A | 2.14 | 2.36 | 1.10 |
| LaLy | A | 2.05 | 6.82 | 3.33 |
| LaPa | A | 1.75 | 6.56 | 3.75 |
| LaAn | A | 1.24 | 3.34 | 2.69 |
| LeSt | A | 1.08 | 2.24 | 2.07 |
| LeMa (2) | A | 0.64 | 1.44 | 2.25 |
| LeMa (3) | A | 0.39 | 1.77 | 4.54 |
| MaAl | A | 3.09 | 3.71 | 1.20 |
| MaMa | A | 1.29 | 3.82 | 2.96 |
| McNa | A | 1.64 | 2.20 | 1.34 |
| MeSa | A | 0.76 | 3.11 | 4.09 |
| MoVe (2) | A | 0.48 | 0.97 | 2.02 |
| OrBe | A | 2.94 | 1.26 | 0.43 |
| PaNa | A | 2.85 | 4.03 | 1.41 |
| SaDj (1) | A | 2.61 | 2.59 | 0.99 |
| SaDa | A | 1.20 | 2.60 | 2.17 |
| ScGi | A | 1.23 | 3.70 | 3.01 |
| ScAn | A | 1.66 | 3.68 | 2.22 |
| SpAn | A | 3.03 | 1.77 | 0.58 |
| StSo | A | 1.51 | 2.13 | 1.41 |
| ToMa | A | 1.82 | 2.19 | 1.20 |
| VaAr | A | 1.38 | 6.06 | 4.39 |
| VaMa | A | 1.39 | 12.96 | 9.32 |
| VaNa (1) | A | 1.97 | 3.10 | 1.57 |
| VaNa (2) | A | 1.54 | 3.44 | 2.23 |
| VeMa | A | 1.05 | 0.77 | 0.73 |
| WePh (1) | A | 1.77 | 16.24 | 9.18 |
| WePh (2) | A | 0.64 | 4.94 | 7.72 |
| BaGr | NA | 0.9 | 0.4 | 0.44 |
| BeMa | NA | 1.17 | 1.85 | 1.58 |
| BeKh | NA | 2.44 | 2.12 | 0.87 |
| DaMa | NA | 6.66 | 12.48 | 1.87 |
| DhAr | NA | 1.35 | 1.85 | 1.37 |
| DiMa | NA | 1.80 | 1.69 | 0.94 |
| DuCa | NA | 2.18 | 1.71 | 0.78 |
| HaMa | NA | 1.81 | 2.09 | 1.15 |
| HaCa | NA | 3.31 | 2.01 | 0.61 |
| HuDo | NA | 1.90 | 1.18 | 0.62 |
| KoMe | NA | 2.45 | 2.70 | 1.10 |
| KrGi | NA | 1.50 | 2.50 | 1.67 |
| LeJo | NA | 1.38 | 1.57 | 1.14 |
| LeMa (1) | NA | 2.04 | 2.94 | 1.44 |
| MoVe (1) | NA | 1.01 | 1.26 | 1.25 |
| PiOu (1) | NA | 3.12 | 0.96 | 0.31 |
| PiOu (2) | NA | 1.52 | 4.45 | 2.92 |
| PoBa | NA | 2.62 | 1.54 | 0.59 |
| SaDj (2) | NA | 2.03 | 2.80 | 1.38 |
| SaJe (1) | NA | 3.23 | 3.07 | 0.95 |
| SaJe (2) | NA | 2.51 | 1.30 | 0.52 |
| SoDo | NA | 1.64 | 2.11 | 1.29 |
| TaFe | NA | 2.45 | 4.78 | 1.95 |
| TaAr | NA | 1.68 | 1.76 | 1.05 |

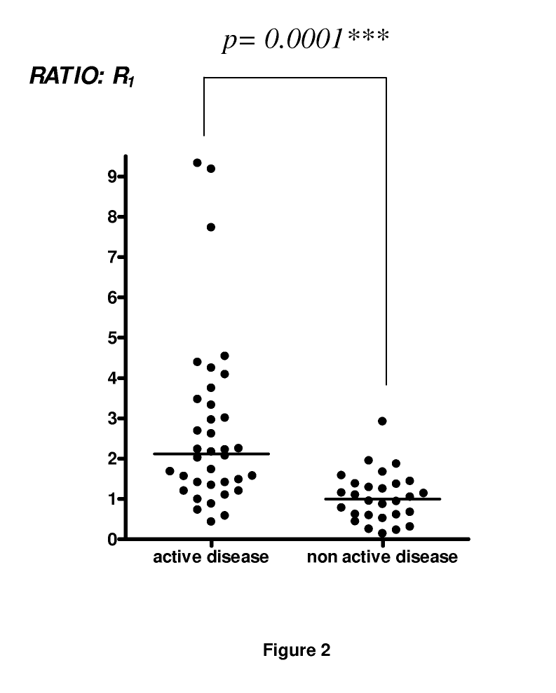

The subject R2 calculated by said equation is clearly higher in patients who are in active state, with a p value of 0.0001 (Mann Witney test), which is seen as highly significant (FIG. 2).

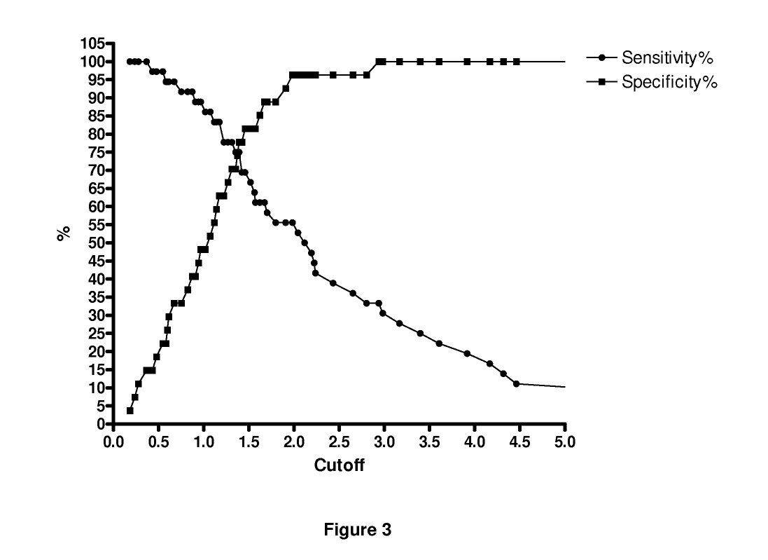

ROC analysis shows that the ratio R1 has a rather poor sensitivity but a very good specificity. For example, for an R1 cut-off value of 2.0, only 53% of the patients will be positive, but those patients that are positive will have 96% of likelihood to develop a relapse (Table 2 and FIG. 3). The sensitivity and specificity percents depending on the cut-off values of R1 are depicted in table 2.

As can be seen from the values in Table 1, two patients (LeMa and MoVe) were analysed both when they were in an active and a non-active state of MS. Comparing the R1-values of these patients in active versus non-active state (1.5 and 1.6 fold increase respectively in the two patients) clearly shows the correlation between the R1-value and disease course. Also, in a patient (LaAn) going from active to non-active MS status, there is a correlating decrease in the R1-value.

TABLE 2

| Cutoff | | |
|---|---|---|
| (R1-value) | Sensitivity (%) | Specificity (%) |
| 0.1850 | 100.000 | 3.704 |
| 0.2400 | 100.000 | 7.407 |
| 0.2800 | 100.000 | 11.110 |
| 0.3700 | 100.000 | 14.810 |
| 0.4350 | 97.220 | 14.810 |
| 0.4800 | 97.220 | 18.520 |
| 0.5500 | 97.220 | 22.220 |
| 0.5850 | 94.440 | 22.220 |
| 0.6000 | 94.440 | 25.930 |
| 0.6150 | 94.440 | 29.630 |
| 0.6750 | 94.440 | 33.330 |
| 0.7550 | 91.670 | 33.330 |
| 0.8250 | 91.670 | 37.040 |
| 0.8750 | 91.670 | 40.740 |
| 0.9100 | 88.890 | 40.740 |
| 0.9450 | 88.890 | 44.440 |
| 0.9700 | 88.890 | 48.150 |
| 1.0200 | 86.110 | 48.150 |
| 1.0750 | 86.110 | 51.850 |
| 1.1200 | 83.330 | 55.560 |
| 1.1450 | 83.330 | 59.260 |
| 1.1750 | 83.330 | 62.960 |
| 1.2250 | 77.780 | 62.960 |
| 1.2700 | 77.780 | 66.670 |
| 1.3150 | 77.780 | 70.370 |
| 1.3550 | 75.000 | 70.370 |
| 1.3750 | 75.000 | 74.070 |
| 1.3950 | 75.000 | 77.780 |
| 1.4250 | 69.440 | 77.780 |
| 1.4600 | 69.440 | 81.480 |
| 1.5200 | 66.670 | 81.480 |
| 1.5650 | 63.890 | 81.480 |
| 1.5750 | 61.110 | 81.480 |
| 1.6250 | 61.110 | 85.190 |
| 1.6750 | 61.110 | 88.890 |
| 1.7050 | 58.330 | 88.890 |
| 1.8000 | 55.560 | 88.890 |
| 1.9100 | 55.560 | 92.590 |
| 1.9850 | 55.560 | 96.300 |
| 2.0450 | 52.780 | 96.300 |
| 2.1200 | 50.000 | 96.300 |
| 2.1950 | 47.220 | 96.300 |
| 2.2250 | 44.440 | 96.300 |
| 2.2400 | 41.670 | 96.300 |
| 2.4350 | 38.890 | 96.300 |
| 2.6550 | 36.110 | 96.300 |
| 2.8050 | 33.330 | 96.300 |
| 2.9400 | 33.330 | 100.000 |
| 2.9850 | 30.560 | 100.000 |
| 3.1700 | 27.780 | 100.000 |
| 3.4000 | 25.000 | 100.000 |
| 3.6100 | 22.220 | 100.000 |

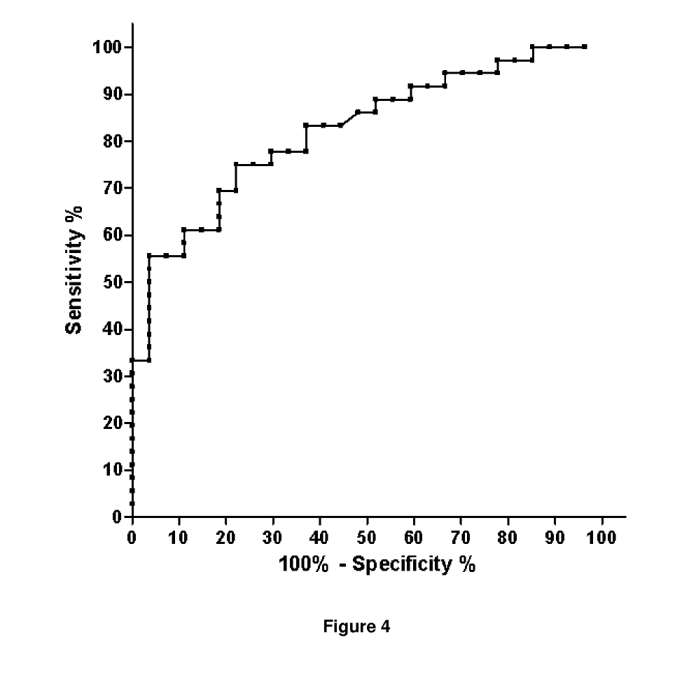

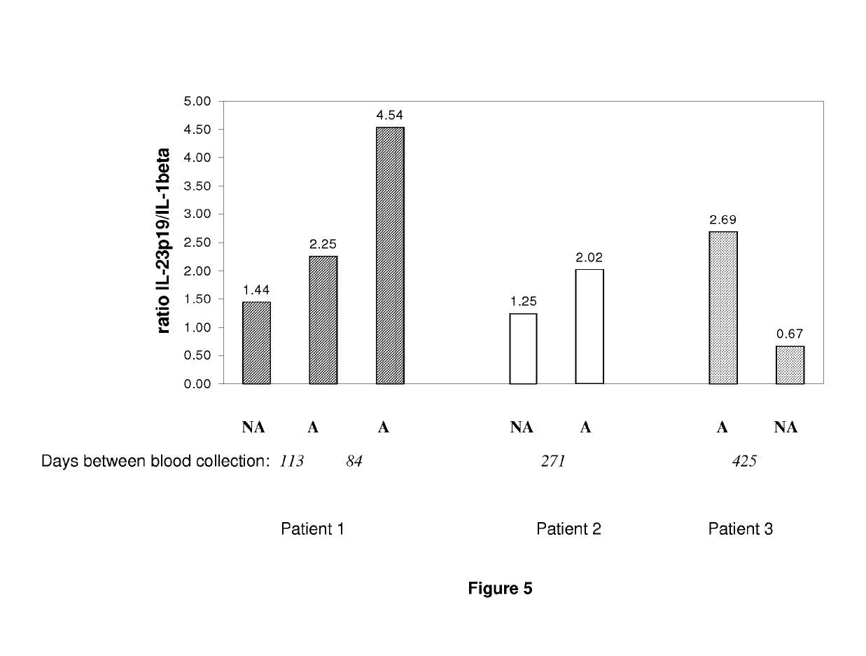

When patients under anti-MS treatment are followed over a certain period, the values of the ratio R1 clearly follow the MS disease activity. FIG. 4 for example shows this. The state of the MS is indicated as NA (non-active) or A (active), as determined by standard diagnostic methods described above. The number of days between taking the blood samples for analysis is also indicated. It is clear that the R1-value is increased in patients that fo from a non-active MS to an active MS state, whereas in patients going from an active to a non-active MS state, the R1-value decreases.

1. A method for predicting, diagnosing and/or prognosticating Multiple Sclerosis (MS) in a subject, comprising the steps of:
(i) measuring the level of IL-23p19 in the sample from the subject before and after stimulation with an immunomodulator, yielding a stimulation index value for IL-23p19;
(ii)
measuring the level of IL-1-beta in the sample from the subject before and after stimulation with an immunomodulator, yielding a stimulation index value for IL-1-beta;
(iii) correlating the relation between the two stimulation indexes obtained in steps (i) and (ii) with respect to each other with the activity status of MS in the subject.

2. The method of claim 1, wherein the correlation step is done using the equation:
R=Stimulation index IL-23p19/Stimulation index IL-1-beta, or any rearrangement thereof.

3. The method of claim 2, wherein the correlation step is done using the equation:
$$R 1 = \frac{\frac{levels of IL \text{-} 23 p 19 after stimulation}{levels of IL \text{-} 23 p 19 before stimulation}}{\frac{levels of IL \text{-} 1 \text{-} beta after stimulation}{levels of IL \text{-} 1 \text{-} beta before stimulation}} \begin{matrix}( Stimulation index IL \text{-} 23 p 19 ) \\ ( Stimulation index IL \text{-} 1 \text{-} beta )\end{matrix}$$
or any rearrangement thereof.

4. The method of claim 3, wherein an increased value of R1 when compared to a reference value is indicative for active MS in the subject.

5. The method according to claim 1, wherein said stimulation with the immunomodulator was done in vivo, by administering the immunomodulator to the subject under investigation, or
wherein said stimulation with the immunomodulator is done in vitro, i.e. by adding the immunomodulator to the sample after it was obtained from the subject.

6. The method according to claim 1, wherein the reference ratio is calculated based on the cytokine levels in a sample from a subject having non-active MS or no MS.

7. The method according to claim 1, wherein the mRNA or protein level of the cytokines is determined.

8. The method according to claim 1, wherein the sample is selected from the group consisting of: blood, whole blood, peripheral blood mononuclear cells (PBMC), plasma or serum.

9. The method according to claim 1, wherein the subject is a human suffering from a disease which can be treated with a type I interferon, multiple sclerosis (MS), (chronic) hepatitis C (HCV) and/or B (HBV).

10. The method according to claim 1, wherein the immunomodulator is selected from the group consisting of: purified or recombinant type-1 interferon (type I IFN), including IFN-alpha and IFN-beta, IFN-alpha-2a, IFN-alpha-2b, IFN-beta-1a, IFN-beta-1b, or agents having similar effects or use in MS such as: Glatiramer Acetate, synthetic polypeptides with a structure resembling myelin, Natalizumab, anti-CD52, anti-CD25, agents acting on the sphingosine receptors such as FTY720, agents having an sequestering effect on lymphocytes or agents depleting T-lymphocytes, Th2-cell response inducing agents such as Fumarate, and wherein the administration of the immunomodulator is performed by intraperitoneal, subcutaneous or intravenous injection or is administered orally.

11. A kit for diagnosing active MS in the subject comprising or consisting of:
(i) means for measuring the level of cytokine IL-23p19 in a sample of the subject;
(ii) means for measuring the level of cytokine IL-1-beta in a sample of the subject;
(iii) optionally an immunomodulator;
(iv) means and/or instructions for calculating the ratio according to the method of claim 1.

12. The kit of claim 11, wherein the means for determining either cytokine level is a means for determining the mRNA or protein level, such as end-point-PCR, real-time-PCR, quantitative-PCR, digital-PCR, or northern blot, capable of determining the mRNA level of the cytokines.

13. The kit of claim 12, wherein the kit comprises:
a) one or more vessel(s) suitable for accepting a blood sample,
b) a primer pair specific to the mRNA of the IL-23p19 gene which is suitable for the transcription of mRNA of said control gene into cDNA and the amplification of the latter, and a probe designed to anneal to an internal region of the produced cDNA,
c) a primer pair specific to the mRNA of the IL-1-beta gene which is suitable for the transcription of mRNA of said control gene into cDNA and the amplification of the latter, and a probe designed to anneal to an internal region of the produced cDNA, wherein said vessel comprises: i) a vessel capable of accepting a blood sample, and optionally ii) a container in which a stabilizing agent is present, iii) a connection between the inside of said vessel (i) and the inside of said container (ii), (iv) a physical barrier that temporarily blocks said connection and optionally (v) a container in which the immunomodulator is present, vi) a connection between the inside of said vessel (i) and the inside of said container (v), and (vii) a physical barrier that temporarily blocks said connection between (i) and (v).

14. The kit of claim 13, wherein the immunomodulator is already present in said vessel (i).

15. The kit of claim 11, wherein the means for determining either cytokine level is a specific binding assay, immunodetection assay, Mass-spectrometry assay, chromatographic assay, capable of determining the protein level of the cytokines.

16. The kit of claim 11, wherein said immunomodulator is purified or recombinant type-1 interferon (type I IFN), including IFN-alpha and IFN-beta, IFN-alpha-2a, IFN-alpha-2b, IFN-beta-1a, IFN-beta-1b, or agents having similar effects or use in MS such as: Glatiramer Acetate, synthetic polypeptides with a structure resembling myelin, Natalizumab, anti-CD52, anti-CD25, agents acting on the sphingosine receptors such as FTY720, agents having an sequestering effect on lymphocytes or agents depleting T-lymphocytes, Th2-cell response inducing agents such as Fumarate.

17. (canceled)

18. The method of claim 1, using a kit according to claim 11.

19. A method for monitoring the treatment of an MS patient comprising performing the method according to claim 1 at different time points during the treatment, wherein reduced ratios point to a reduction in MS activity in the subject under treatment, indicating the treatment is indeed beneficial for the subject.

20. A method for determining the treatment needed for an MS patient comprising performing the method according to claim 1 at different time points during the treatment, wherein increased ratios point to active MS in the subject under observation, indicating the need for active MS specific treatment.
